(19) United States
(12) Patent Application Publication
(10) Pub. No.: US 2026/0245662 A1
(43) Pub. Date: Aug. 20, 2026

(54) SYSTEM AND METHOD FOR NON-INVASIVE GENETIC MAPPING

(71) Applicant: Austin A. Forman, Parma, OH (US)

(72) Inventor: Austin A. Forman, Parma, OH (US)

(21) Appl. No.: 19/053,496

(22) Filed: Feb. 14, 2025

(51) Int. Cl. G16B 40/10; G06V 20/69; G16B 25/10; G16B 45/00

(52) U.S. Cl. CPC G16B 40/10; G06V 20/698; G16B 25/10; G16B 45/00

(57) ABSTRACT

Systems and methods for non-invasive genetic mapping using artificial intelligence are provided. A mobile device captures or receives image data of a target organism and transmits the data to a remote artificial intelligence system. The Al system analyzes the image data to extract genetic markers, compares the markers to reference databases, identifies genes and mutations, and maps them to specific gene loci. A comprehensive genetic map is generated and transmitted back to the mobile device for display. This approach enables genetic mapping and analysis without requiring invasive tissue sampling procedures.

### CROSS-REFERENCE TO RELATED APPLICATIONS

This application claims the benefit of U.S. Provisional Application No. [NUMBER], filed [DATE], which is incorporated herein by reference in its entirety.

### FIELD OF THE INVENTION

The present disclosure relates generally to genetic analysis systems and methods. More particularly, the present disclosure relates to systems and methods for non-invasive genetic mapping and analysis using artificial intelligence.

### BACKGROUND

Genetic mapping and analysis traditionally requires invasive tissue sampling procedures to obtain genetic material for sequencing and analysis. These conventional methods can be time- consuming, expensive, and carry risks of contamination and human error. There is a need for improved systems and methods that can perform genetic mapping and analysis in a non-invasive manner.

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### BRIEF SUMMARY

The present disclosure provides systems and methods for non-invasive genetic mapping and analysis using artificial intelligence. In some embodiments, a mobile application enables users to capture or upload images or videos of organisms within the Kingdom Animalia. An artificial intelligence system analyzes the image/video data to identify genes and genetic mutations, mapping them to specific gene loci. This allows for generation of a comprehensive genetic map without requiring invasive tissue sampling.

In one aspect, a system for non-invasive genetic mapping is provided. The system may include a mobile device with an image capture component, a processor, and a memory storing instructions. When executed, the instructions may cause the processor to: receive image data of a target organism captured by the image capture component; preprocess the image data; transmit the preprocessed image data to a remote artificial intelligence system; receive genetic analysis results from the artificial intelligence system, the results including identified genes and genetic mutations mapped to specific gene loci; and display the genetic analysis results on a display of the mobile device.

In another aspect, a method for non-invasive genetic mapping is provided. The method may include: receiving, at an artificial intelligence system, image data of a target organism from a mobile device; applying one or more artificial intelligence algorithms to extract genetic markers from the image data; comparing the extracted genetic markers to one or more genetic reference databases; identifying genes and genetic mutations based on the comparison; mapping the identified genes and genetic mutations to specific gene loci; generating a genetic map based on the mapped genes and mutations; and transmitting the genetic map to the mobile device for display.

Further areas of applicability of the present disclosure will become apparent from the detailed description provided hereinafter. It should be understood that the detailed description and specific examples, while indicating various embodiments, are intended for purposes of illustration only and are not intended to necessarily limit the scope of the disclosure.

### BRIEF DESCRIPTION OF THE DRAWINGS

The present disclosure will become more fully understood from the detailed description and the accompanying drawings, wherein:

FIG. 1 is a block diagram illustrating an example system for non-invasive genetic mapping using artificial intelligence in accordance with embodiments of the present disclosure;

FIG. 2 is a flowchart illustrating an example method for non-invasive genetic mapping from the perspective of a mobile device in accordance with embodiments of the present disclosure;

FIG. 3 is a flowchart illustrating an example method for non-invasive genetic mapping from the perspective of an artificial intelligence backend system in accordance with embodiments of the present disclosure;

FIG. 4 is a diagram illustrating an example process flow for genetic data transformation in accordance with embodiments of the present disclosure; and

FIG. 5 is a screenshot illustrating an example user interface displaying genetic mapping results on a mobile device in accordance with embodiments of the present disclosure.

The drawings described herein are for illustrative purposes only of selected embodiments and not all possible implementations, and are not intended to limit the scope of the present disclosure. Corresponding reference numerals indicate corresponding parts throughout the several views of the drawings.

### DETAILED DESCRIPTION

Example embodiments will now be described more fully with reference to the accompanying drawings. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure.

The terminology used herein is for the purpose of describing particular example embodiments only and is not intended to be limiting. As used herein, the singular forms "a," "an," and "the" may be intended to include the plural forms as well, unless the context clearly indicates otherwise. The terms "comprises," "comprising," "including," and "having," are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being "on," "engaged to," "connected to," or "coupled to" another element or layer, it may be directly on, engaged, connected or coupled to the other element or layer, or intervening elements or layers may be present. In contrast, when an element is referred to as being "directly on," "directly engaged to," "directly connected to," or "directly coupled to" another element or layer, there may be no intervening elements or layers present. Other words used to describe the relationship between elements should be interpreted in a like fashion (e.g., "between" versus "directly between," "adjacent" versus "directly adjacent," etc.). As used herein, the term "and/or" includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as "first," "second," and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as "inner," "outer," "beneath," "below," "lower," "above," "upper," and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as "below" or "beneath" other elements or features would then be oriented "above" the other elements or features. Thus, the example term "below" can encompass both an orientation of above and below. The device may be otherwise oriented (rotated 90 degrees or at other orientations) and the spatially relative descriptors used herein interpreted accordingly.

The present disclosure provides systems and methods for non-invasive genetic mapping and analysis using artificial intelligence. In various embodiments, a mobile application enables users to capture or upload images or videos of organisms within the Kingdom Animalia. An artificial intelligence system analyzes the image/video data to identify genes and genetic mutations, mapping them to specific gene loci. This allows for generation of a comprehensive genetic map without requiring invasive tissue sampling.

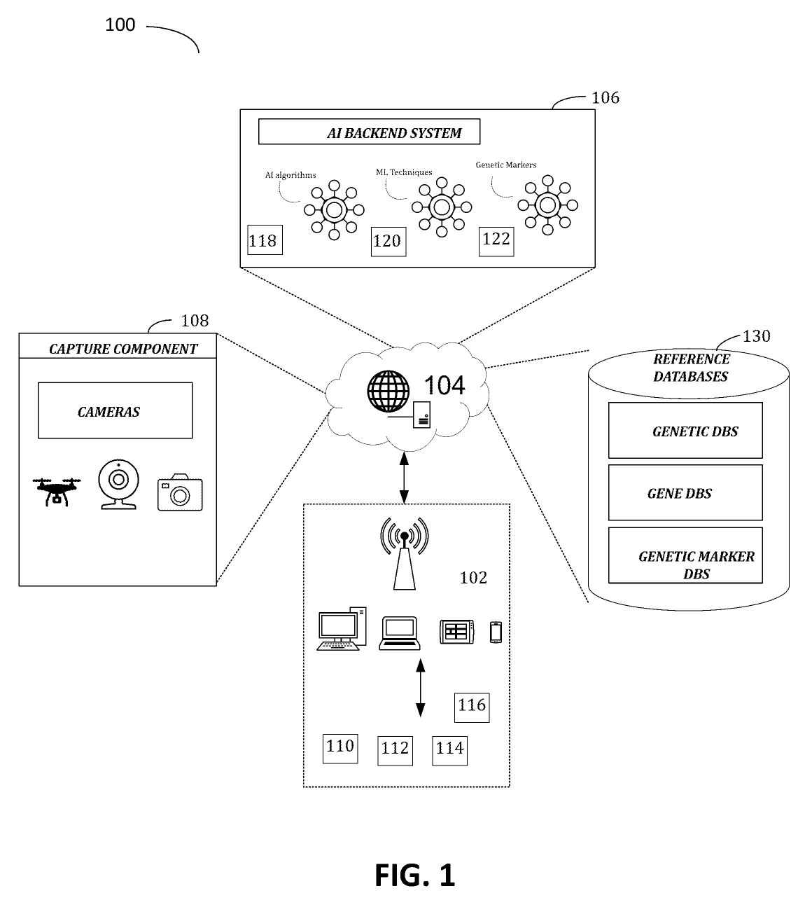

FIG. 1 illustrates an example system 100 for non-invasive genetic mapping using artificial intelligence in accordance with embodiments of the present disclosure. The system 100 may include a mobile device 102, a network 104, and an artificial intelligence (AI) backend system 106.

The mobile device 102 may be any suitable portable computing device, such as a smartphone, tablet, laptop computer, or wearable device. The mobile device 102 may include an image capture component 108, such as a camera, for capturing images or videos of target organisms. The mobile device 102 may also include a processor 110, a memory 112, a display 114, and a network interface 116.

The memory 112 may store instructions that, when executed by the processor 110, cause the mobile device 102 to perform various operations related to non-invasive genetic mapping. These operations may include capturing or receiving image/video data, preprocessing the data, transmitting the data to the AI backend system 106, receiving genetic analysis results, and displaying the results on the display 114.

The network 104 may be any suitable communication network or combination of networks, such as cellular networks, Wi-Fi networks, or the Internet, that enable communication between the mobile device 102 and the AI backend system 106.

The AI backend system 106 may include one or more servers or cloud-based computing resources configured to perform artificial intelligence-based analysis of image/video data for genetic mapping purposes. The AI backend system 106 may include a processor 118, a memory 120, and a network interface 122.

The memory 120 may store instructions that, when executed by the processor 118, cause the AI backend system 106 to perform various operations related to genetic analysis and mapping. These operations may include receiving image/video data from mobile devices, applying AI algorithms to extract genetic markers, comparing extracted data to genetic reference databases 130, identifying genes and mutations, mapping to specific gene loci, generating comprehensive genetic maps, and returning results to mobile devices.

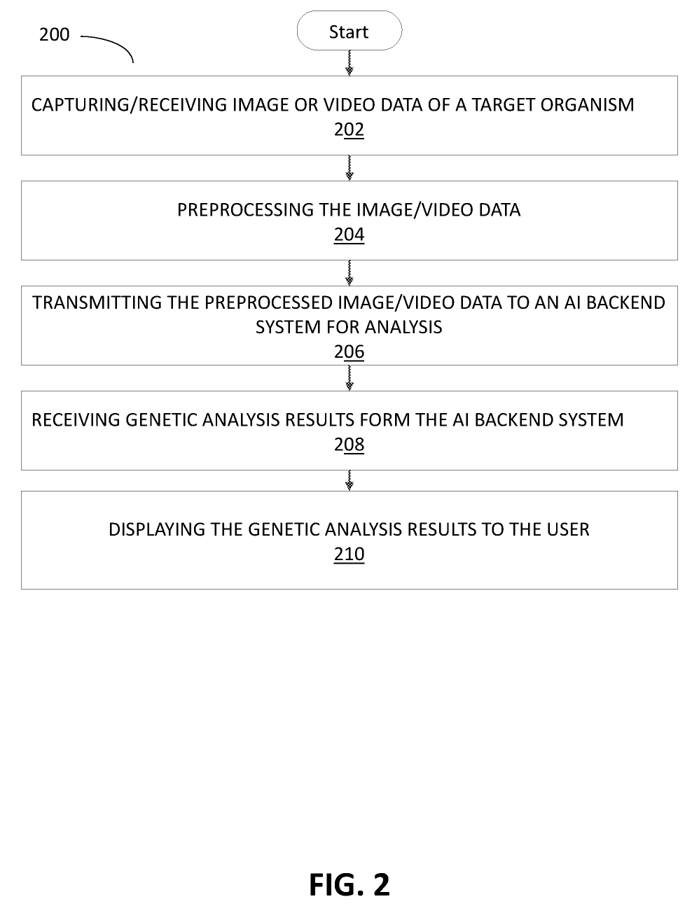

FIG. 2 is a flowchart illustrating an example method 200 for non-invasive genetic mapping from the perspective of a mobile device in accordance with embodiments of the present disclosure. The method 200 may be performed by a mobile device, such as the mobile device 102 shown in FIG. 1.

At step 202, the mobile device may capture or receive image or video data of a target organism. This may involve using an integrated camera to capture new images/videos or receiving previously captured images/videos uploaded by a user.

At step 204, the mobile device may preprocess the image/video data. This preprocessing may include various image processing techniques to enhance image quality, remove noise, or extract relevant features.

At step 206, the mobile device may transmit the preprocessed image/video data to an AI backend system for analysis. This transmission may occur over a network connection, such as a cellular or Wi-Fi network.

At step 208, the mobile device may receive genetic analysis results from the AI backend system. These results may include identified genes, genetic mutations, and their mappings to specific gene loci.

At step 210, the mobile device may display the genetic analysis results to the user. This may involve presenting the results in a user-friendly format on the device's display, potentially including visualizations of the genetic map.

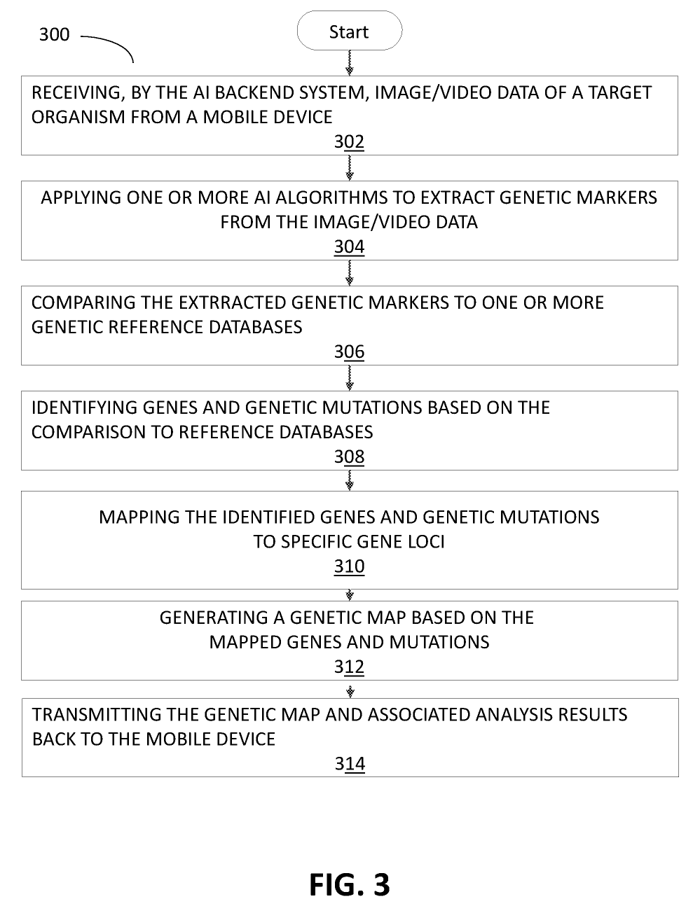

FIG. 3 is a flowchart illustrating an example method 300 for non-invasive genetic mapping from the perspective of an artificial intelligence backend system in accordance with embodiments of the present disclosure. The method 300 may be performed by an AI backend system, such as the AI backend system 106 shown in FIG. 1.

At step 302, the AI backend system may receive image/video data of a target organism from a mobile device. This data may have been preprocessed by the mobile device before transmission.

At step 304, the AI backend system may apply one or more AI algorithms to extract genetic markers from the image/video data. These algorithms may utilize various machine learning techniques, such as convolutional neural networks or computer vision algorithms, to identify relevant genetic features from visual data.

At step 306, the AI backend system may compare the extracted genetic markers to one or more genetic reference databases. These databases may contain known genetic information for various organisms within the Kingdom Animalia.

At step 308, the AI backend system may identify genes and genetic mutations based on the comparison to reference databases. This may involve pattern matching, statistical analysis, and other computational techniques to determine the most likely genetic makeup of the target organism.

At step 310, the AI backend system may map the identified genes and genetic mutations to specific gene loci. This mapping process creates a comprehensive picture of the organism's genetic structure.

At step 312, the AI backend system may generate a genetic map based on the mapped genes and mutations. This map may provide a visual or data-driven representation of the organism's genetic makeup.

At step 314, the AI backend system may transmit the genetic map and associated analysis results back to the mobile device for display to the user.

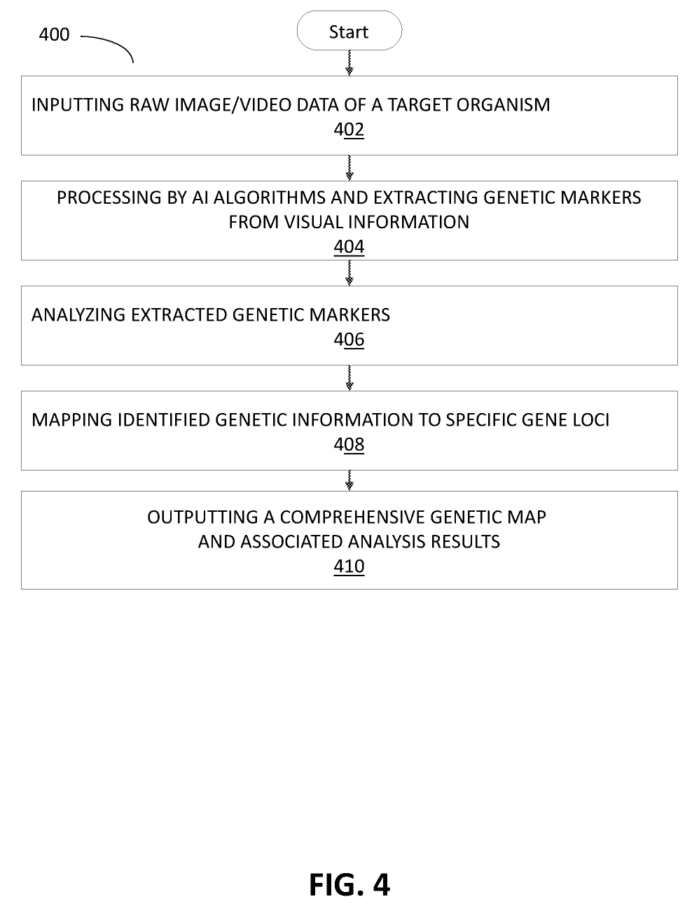

FIG. 4 is a diagram illustrating an example process flow 400 for genetic data transformation in accordance with embodiments of the present disclosure. The process flow 400 demonstrates how raw image/video data is transformed into a comprehensive genetic map through AI-powered analysis.

The process begins with input 402, which consists of raw image/video data of a target organism. This data may be captured by a mobile device or received from an external imaging device.

The raw data undergoes AI-based processing 404, where artificial intelligence algorithms extract genetic markers from the visual information. This step may involve complex image analysis techniques to identify relevant genetic features.

The extracted genetic markers then undergo analysis 406, where they are compared to reference databases to identify specific genes and potential mutations. This step may involve large-scale data comparisons and statistical analysis.

The identified genetic information is then mapped 408 to specific gene loci, creating a detailed picture of the organism's genetic structure. This mapping process associates genetic features with their physical locations on chromosomes.

Finally, the process produces output 410 in the form of a comprehensive genetic map and associated analysis results. This output may include visualizations, data tables, and detailed genetic information about the target organism.

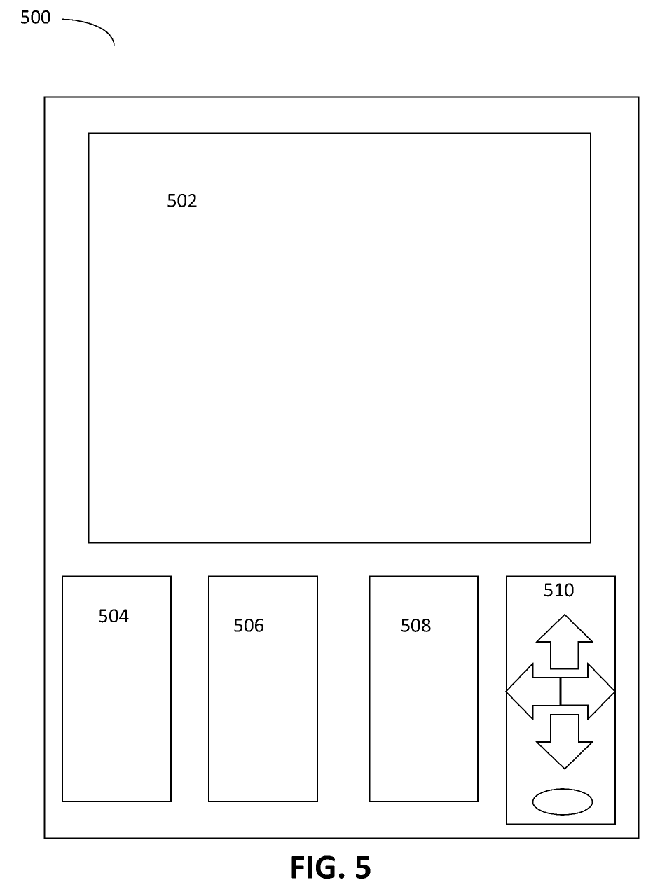

FIG. 5 is a screenshot illustrating an example user interface 500 displaying genetic mapping results on a mobile device in accordance with embodiments of the present disclosure. The user interface 500 may be presented on the display of a mobile device after receiving analysis results from the AI backend system.

The user interface 500 may include an image display area 502 showing the original image or video of the target organism. A genetic map visualization 504 may present a graphical representation of the organism's genetic structure, with identified genes and mutations highlighted.

A results summary section 506 may provide an overview of key findings from the genetic analysis. This may include information about identified genetic traits, potential health implications, or other relevant data.

A detailed results section 508 may allow users to explore specific genes or mutations in greater depth. This section may provide additional context, explanations, or links to further resources about the genetic information.

Navigation controls 510 may enable users to interact with the results, such as zooming in on specific areas of the genetic map or switching between different views of the data.

The systems and methods described herein provide several advantages over traditional genetic mapping techniques. By utilizing artificial intelligence to analyze visual data, the need for invasive tissue sampling is eliminated. This non-invasive approach reduces risks associated with sample collection and processing, while potentially increasing the speed and accessibility of genetic analysis.

The mobile application interface allows users to easily capture or upload images/videos for analysis, making genetic mapping more accessible to a wider range of users. The AI-powered backend system can process large amounts of visual data quickly and accurately, potentially identifying genetic markers that may be missed by human analysis.

The comprehensive genetic maps generated by the system can provide valuable insights into an organism's genetic makeup, potentially identifying mutations or genetic traits that may be relevant for research, breeding programs, or medical diagnosis.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A system for non-invasive genetic mapping, comprising:
a mobile device comprising:
an image capture component;
a processor; and
a memory storing instructions that, when executed by the processor, cause the processor to:
receive image data of a target organism captured by the image capture component;
preprocess the image data;
transmit the preprocessed image data to a remote artificial intelligence system;
receive genetic analysis results from the artificial intelligence system, the results including identified genes and genetic mutations mapped to specific gene loci; and display the genetic analysis results on a display of the mobile device.

2. The system of claim 1, wherein the target organism is within the Kingdom Animalia.

3. The system of claim 1, wherein the image data comprises at least one of a still image or a video.

4. The system of claim 1, further comprising:
the remote artificial intelligence system, wherein the remote artificial intelligence system comprises:
a processor; and
a memory storing instructions that, when executed by the processor, cause the processor to:
receive the preprocessed image data from the mobile device;
apply one or more artificial intelligence algorithms to extract genetic markers from the preprocessed image data;
compare the extracted genetic markers to one or more genetic reference databases;
identify genes and genetic mutations based on the comparison;
map the identified genes and genetic mutations to specific gene loci;
generate a genetic map based on the mapped genes and mutations; and transmit the genetic map to the mobile device for display.

5. The system of claim 4, wherein the one or more artificial intelligence algorithms comprise at least one of a convolutional neural network or a computer vision algorithm.

6. The system of claim 1, wherein the mobile device further comprises a network interface for communicating with the remote artificial intelligence system.

7. The system of claim 1, wherein the instructions further cause the processor to:
receive previously captured image data uploaded by a user; and transmit the uploaded image data to the remote artificial intelligence system for analysis.

8. A method for non-invasive genetic mapping, comprising:
receiving, at an artificial intelligence system, image data of a target organism from a mobile device;
applying one or more artificial intelligence algorithms to extract genetic markers from the image data;
comparing the extracted genetic markers to one or more genetic reference databases;
identifying genes and genetic mutations based on the comparison;
mapping the identified genes and genetic mutations to specific gene loci;
generating a genetic map based on the mapped genes and mutations; and transmitting the genetic map to the mobile device for display.

9. The method of claim 8, wherein the target organism is within the Kingdom Animalia.

10. The method of claim 8, wherein the image data comprises at least one of a still image or a video.

11. The method of claim 8, wherein the one or more artificial intelligence algorithms comprise at least one of a convolutional neural network or a computer vision algorithm.

12. The method of claim 8, further comprising:
preprocessing the image data at the mobile device prior to transmitting the image data to the artificial intelligence system.

13. The method of claim 8, further comprising:
displaying the genetic map on a display of the mobile device.

14. The method of claim 13, wherein displaying the genetic map comprises presenting a user interface including at least one of:
an image display area showing the image data of the target organism;
a genetic map visualization;
a results summary section; or a detailed results section.

15. A non-transitory computer-readable medium storing instructions that, when executed by a processor of a mobile device, cause the processor to perform operations comprising:
capturing image data of a target organism using an image capture component of the mobile device;
preprocessing the image data;
transmitting the preprocessed image data to a remote artificial intelligence system;
receiving genetic analysis results from the artificial intelligence system, the results including identified genes and genetic mutations mapped to specific gene loci; and displaying the genetic analysis results on a display of the mobile device.

16. The non-transitory computer-readable medium of claim 15, wherein the target organism is within the Kingdom Animalia.

17. The non-transitory computer-readable medium of claim 15, wherein the image data comprises at least one of a still image or a video.

18. The non-transitory computer-readable medium of claim 15, wherein the operations further comprise:
receiving previously captured image data uploaded by a user; and transmitting the uploaded image data to the remote artificial intelligence system for analysis.

19. The non-transitory computer-readable medium of claim 15, wherein displaying the genetic analysis results comprises presenting a user interface including at least one of:
an image display area showing the image data of the target organism;
a genetic map visualization;
a results summary section; or a detailed results section.

20. The non-transitory computer-readable medium of claim 15, wherein the operations further comprise:
receiving user input to interact with the displayed genetic analysis results; and updating the display based on the user input.
